(19) United States
(12) Patent Application Publication
(10) Pub. No.: US 2004/0053895 A1
(43) Pub. Date: Mar. 18, 2004

(54) Multi-use vessels for vitamin D formulations

(75) Inventors: Richard B. Mazess, Madison, WI (US); Jeffrey W. Driscoll, Middleton, WI (US); Creighton Reed Goldensoph, DeForest, WI (US); Leon W. LeVan, Oregon, WI (US)

Correspondence Address: MICHAEL BEST & FRIEDRICH, LLP, ONE SOUTH PINCKNEY STREET, P O BOX 1806, MADISON, WI, 53701

(73) Assignee: Bone Care International, Inc., Middleton, WI

(21) Appl. No.: 10/247,766

(22) Filed: Sep. 18, 2002

(51) Int. Cl. A61K 31/59; A61M 31/00

(52) U.S. Cl. 514/167; 604/500

(57) ABSTRACT

This invention relates to multi-use dispensing vessels containing pharmaceutical formulations of active vitamin D compounds, and also to plastic blow fill containers containing active vitamin D formulations.

### CROSS-REFERENCE TO RELATED APPLICATIONS

Not Applicable

### STATEMENT REGARDING FEDERALLY SPONSORED RESEARCH OR DEVELOPMENT

Not Applicable

### BACKGROUND OF THE INVENTION

This invention relates to multi-use dispensing vessels or vials containing pharmaceutical formulations of vitamin D compounds and analogs. The multi-use vials are particularly useful for parenteral administration. The invention also relates to plastic blow-fill containers containing pharmaceutical formulations of vitamin D compounds and analogs.

For intravenous administration, drug formulations of, e.g., vitamin D compounds or analogs, are routinely supplied in single-use, single-dose glass ampoules or vials (glass vessels with a rubber top) for several reasons. Ampoules were the first containers used in an attempt to prevent contamination and oxidation of the active ingredients. The need to protect active compounds also led to the routine addition of antioxidants to preserve the integrity of the active agent. An example of a widely used antioxidant is ascorbic acid or sodium ascorbate, which is put into aqueous solutions of the active agent together with a surfactant to facilitate mixture. Drug formulations also have often been stabilized with buffers, such as acetate, citrate, glutamate, and phosphate buffers, to maintain pH, and chelating agents, such as citric acid, tartaric acid, amino acids, thioglycolic acid, and ethylenediamine tetraacetic acid disodium (EDTA). Subsequently, aqueous solutions have been replaced with solutions containing co-solvents, such as propylene glycol and an alcohol, in addition to water (See, U.S. Pat. No. 6,136,799 issued to Li et al). This combination has allowed certain drug compounds to be stable in glass vials. Despite these various developments in single-use, single-dose ampoule or vial-contained formulations, there is an inherent increased cost because the ampoules or vials are disposed of after the single use. Further, they remain costly to fabricate, are subject to breakage, and require a syringe and needle to load and inject the formulation.

Some cost savings are potentially available with multi-use vessels or vials. Multi-use vessels are suitable for multiple administrations in any desired partial amounts of a solution of an active substance. A multi-use vial makes dosing more efficient, minimizes wasted product, contains costs, and makes better use of storage space. In some clinic settings where multiple patients are treated with the same formulation, a formulation suitable for a multi-use vial could provide significant savings, e.g., in reduced number and cost of containers.

However, special demands are made on the stability, sterility and storability of such forms of administration. Multi-use vials, which require a syringe and needle be loaded with each dose, have a risk of contamination from reuse of the needle or syringe, or improper decontamination of the rubber membrane. Reports of infections transmitted through contaminated multidose vials clearly suggest that their use poses a tangible risk. Further, while common antimicrobial preservatives used in multidose vials may be highly effective for most bacteria, they are typically not antiviral agents. Also, there appears to be a vulnerable window of time (about two hours) during which contaminating organisms may remain viable in multidose vials before the preservative fully exerts its effect. Even after the preservative inactivates the organism, endotoxins may be present and can cause pyretic or febrile reactions. While faulty aseptic technique is often the primary cause of vial contamination and significant microbial population, other factors include the design of the vial, storage conditions, the frequency of entering the vial, the environmental air injected into the vial and, of course, the nature of the drug formulation itself.

Needleless systems are now available for removal of the contents of multi-use vials, and can virtually eliminate the needle reuse, reentry risk. Because of multiple withdrawals from a vial, storage problems, etc. most existing formulations are neither suitable for nor approved for multi-use vials even with needleless systems.

New types of plastic containers (blow-fill) for intravenous drugs have also been developed which are of much lower cost and not subject to breakage. Moreover, these containers also can be made with a luer lock entry, thereby enabling them to be connected directly to a syringe or an indwelling catheter in a patient. This direct administration eliminates the need for a needle and syringe to withdraw and administer a dose. However, the existing formulations for vitamin D analogs do not allow these plastic containers to be used. Plastic containers are permeable to oxygen. A simple aqueous solution with an antioxidant is typically not stable, and the antioxidant, in some cases, is subject to degradation and/or discoloration. Also, a solution containing alcohol is likely to be unstable because the alcohol may interact chemically with the plastic.

### BRIEF SUMMARY OF INVENTION

The present invention provides multi-use vials containing an active vitamin D formulation. The invention also provides plastic blow-fill containers containing a vitamin D formulation. The vitamin D formulation in accordance with the present invention comprises 1) an active vitamin D compound or analog, 2) a non-ionic solubilizer, 3) a lipophilic antioxidant, and 4) optionally, an agent(s) that is an organic solvent, a preservative or both, in an aqueous vehicle. The formulation of present invention, thus, comprises a therapeutically effective amount of a vitamin D compound or analog, a non-ionic solubilizer, a small amount of lipophilic antioxidant, and optionally, an agent that includes an organic solvent (e.g., ethanol) or co-solvents (e.g., propylene glycol and ethanol) and/or a preservative (e.g., benzyl alcohol).

The formulations of the present invention may be formulated in a variety of concentrations in various vial sizes for various administration dosages. The concentrations will depend on the particular vitamin D compound or analog used and the nature of the desired therapeutic response. For example, a formulation in accordance with the present invention may comprise a 5 μg dose of doxercalciferol. Additionally, the dosage may be formulated in a 4 mL or an 8 mL vial, or any other size vial or container suitable for the formulation.

The invention also relates to methods for the treatment and prevention of certain diseases and disorders comprising employing a multi-use vessel or plastic blow-fill vessel containing a vitamin D formulation in accordance with the present invention to administer to a patient in need thereof such formulation. These diseases include hyperparathyroidism, e.g., secondary hyperparathyroidism, neoplastic diseases, such as cancers of the pancreas, breast, colon or prostate as well as other diseases of abnormal cell differentiation and/or cell proliferation such as psoriasis, and disorders of calcium metabolism such as osteomalacia.

Other advantages and a fuller appreciation of the specific attributes of this invention will be gained upon an examination of the following detailed description of the invention, and appended claims.

### BRIEF DESCRIPTION OF THE DRAWINGS

Not applicable.

### DETAILED DESCRIPTION OF INVENTION

The present invention provides multi-use vials and plastic blow-fill containers that comprise a vitamin D formulation. The pharmaceutical formulation comprises a therapeutically effective amount of an active vitamin D compound or analog, a non-ionic solubilizer, and a lipophilic antioxidant, and optionally, an agent that includes an organic solvent, a preservative or both, in an aqueous vehicle. An illustrated embodiment of the multi-use vessel includes a volume greater than 2 mL and contains greater than 2 mL of the vitamin D formulation.

As used herein, the term “activated vitamin D” or “active vitamin D” in reference to a compound is intended to include any biologically active vitamin D compound, including a pro-drug (or pro-hormone), a precursor, a metabolite or an analog, in any stage of its metabolism. It is known that vitamin D compounds display a variety of biological activities, e.g., in calcium and phosphate metabolism (see, e.g., U.S. Pat. No. 5,104,864), as an antineoplastic agent (see, e.g., U.S. Pat. No. 5,763,429), and as an anti-hyperparathyroid agent (see, e.g., U.S. Pat. No. 5,602,116). It is contemplated that any of the biologically active forms of vitamin D can be used in the formulations in accordance with the present invention. Generally, an active vitamin D compound or analog is hydroxylated in at least the C-1, C-24 or C-25 position of the molecule, and either the compound itself or its metabolite binds to the vitamin D receptor (VDR). Pro-drugs, for example, include vitamin D compounds that are hydroxylated in the C-1. Such compounds undergo further hydroxylation in vivo, and their metabolites bind the VDR.

Precursors include previtamins, such as α-hydroxyprevitamin D₂, 1α,24-dihydroxyprevitamin D₂, 1α,25-dihydroxyprevitamin D₂, 24-hydroxyprevitamin D₂, 1α-hydroxyprevitamin D₃ and 1α,25-dihydroxyprevitamin D₃, which are thermal isomeric forms of the vitamin forms. Metabolites generally include compounds or analogs that have undergone further metabolic processing, e.g., hydroxylation.

Examples of compounds suitable for formulations of the present invention include, without limitation, 1α,24-dihydroxyvitamin D₂, 1α,2-dihydroxyvitamin D₄, 1α,24-dihydroxyvitamin D₂, 1α, 25-dihydroxyvitamin D₃ (calcitriol), 1α,25-dihydroxyvitamin D₂, 1α,25-dihydroxyvitamin D₄, α, 24,25-dihydroxyvitamin D₂, 1α-hydroxyvitamin D₃ (α-calcidol), seocalcitol (EB-1089), calcipotriol, 22-oxacalcitriol (maxacalcitol), fluorinated compounds such as falecalcitriol, and 19-nor compounds such as paricalcitol. Among those compounds that have a chiral center, e.g., in the sidechain, such as at C-24, it is understood that both epimers (e.g., R and S) and the racemic mixture are within the scope of the present invention.

As used herein, the term “organic solvent” is also meant to include co-solvents, e.g., a combination of propylene glycol and ethanol.

As used herein, the term “preservative” is meant to refer to an antimicrobial agent, e.g., an anti-bacterial agent.

It also is understood that any numerical value recited herein includes all values from the lower value to the upper value. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between the lowest value and the highest value enumerated are to be considered to be expressly stated in this application.

The amount of selected vitamin D compound or analog is not critical to the present invention and may be varied to achieve the desired therapeutic response for a particular patient. The amount of active vitamin D in the formulations of the invention will be, in part, dependent on the solubility of the specific solubilizer used. Those skilled in the arts can adjust the ratios without undue experimentation. The selected dosage also will depend on the activity of the specific compound or analog, the route of administration, the severity of the condition being treated, and the condition and history of the specific patient. For example, a therapeutic dose for active vitamin D compounds may range between about 2 μg and about 100 μg per dose.

Suitable solubilizing agents for the formulations of the present invention include non-ionic solubilizers. A non-ionic solubilizer is one where the hydrophilic part of the solubilizer carries no charge but derives its water solubility from highly polar groups such as hydroxyl or polyoxyethylene groups. Some surfactants known for use in the pharmaceutical field also have a solubilizing function.

Solubilizers generally include, without limitation, polyoxyalkylenes, cyclodextrins, dextrans, fatty acid esters of saccharose, fatty alcohol ethers of oligoglucosides (e.g., the akylpolyglucosides such as TRITON™), fatty acid esters of glycerol (as, e.g., glycerol mono/distearate or glycerol monolaurate), polyoxyethylene type compounds (PEGs, SOLUTOL™s, CREOMOPHOR™s, POE, PEO, Macrogol, Carbowax, Polyoxyl). The latter also include polyethoxylated fatty acid esters of sorbitan (e.g., polysorbates, such as TWEEN™s, “SPAN™s), fatty acid esters of poly(ethylene oxide) (e.g., polyoxyethylene stearates), fatty alcohol ethers of poly(ethylene oxide) (e.g., polyoxyethylated lauryl ether), alkylphenol ethers of poty(ethylene oxide) (e.g., polyethoxylated octylphenol), polyoxyethylene-polyoxypropylene block copolymers (also known as poloxamers, such as “Pluronic”), and ethoxylated fats and oils (e.g., ethoxylated castor oil, or polyoxyethylated castor oil (also known as polyethylene glycol-glyceryl triricinoleate). Mixtures of solublilizers are also within the scope of the invention. Such mixtures are readily available from standard commercial sources. Solubilizers of particular interest include polysorbates, e.g. TWEEN™. The amount of such solubilizer present in the formulations of the present invention includes from about 0.05% to about 5% w/w.

Suitable lipophilic antioxidants include, without limitation, butylated hydroxytoluene (BHT), lipoic acid, lycopene, lutein, lycophyll, xanthophyll, carotene, zeaxanthin or vitamin E and/or esters thereof. The lipophilic antioxidants are present in very small but effective amounts, e.g., about 20 to about 2000 ppm.

If desired, formulations of the present invention can include optional agents. Examples of such agents include, without limitation, agents that are organic solvents, preservatives or both. Such agents include alcohols and polyols, such as ethanol, benzyl alcohol, isopropanol, butanol, ethylene glycol, propylene glycol, butanediols, glycerol, pentaerythritol, sorbitol, mannitol, transcutol, dimethyl isosorbide, polyethylene glycol, polypropylene glycol, polyvinylalcohol, hydroxypropyl methylcellulose and other cellulose derivatives, cyclodextrins and cyclodextrin derivatives. Amounts of optional agents include 0% to 30% w/w, e.g., organic solvent.

Multi-use vessels in accordance with the present invention generally include formulations of an active vitamin D compound, 0.05% to 5% w/w of a non-ionic solubilizer (e.g., a polysorbate), 20 to 2000 ppm lipophilic antioxidant (e.g., BHT), and optionally, 0% to 30% of an agent that is an organic solvent (or co-solvents), preservative, or both (e.g., benzyl alcohol).

A multi-use vessel for use in treating secondary hyperparathyroidism suitably includes a particular formulation that contains 2-6 μg/mL 1α-hydroxyvitamin D₂ (doxercalciferol), 2.5% w/w benzyl alcohol, 0.5%-2.5% w/w TWEEN™-20, and 20 ppm BHT. Another formulation in accordance with the present invention suitable for treating secondary hyperparathyroidism includes paricalcitol. Yet another suitable formulation includes 1α,25-dihydroxyvitamin D₃ (calcitriol).

A pharmaceutical formulation in accordance with the present invention comprises an aqueous vehicle. The aqueous vehicle contains, of course, water, but it may furthermore also contain pH adjusting agents, stabilizing agents, solubilizing agent (see, hereinabove), isotonic adjusting agents, and solvents (e.g. organic solvents; as discussed above). The formulations in accordance with the present invention preclude the need for high organic solvent, which can cause irritation in some patients. In some cases, however, it may be appropriate to include an organic solvent or co-solvent, for example, in some cases up to 30% ethanol. The amount of water in a formulation in accordance with the present invention is normally at least about from about 50% to about 99% w/w.

For the pharmaceutical formulations contained in the multi-use vessels of the present invention, the intended route of administration is suitably parenteral, i.e., for use by injection into, e.g., an animal or human body. Such route includes intravenous, intramuscular and subcutaneous administration, the intravenous route being especially suitable for the formulations of the present invention for use in connection with, e.g., secondary hyperparathyroidism or neoplastic disorders.

It is also understood the formulation in accordance with the present invention may be contained in a vial, bottle, tube syringe or other container for multiple administrations. Such containers may be made of glass or a polymer material such as polypropylene, polyethylene, or polyvinylchloride. Container may include a seal, or other closure system, such as a rubber stopper, or may be part of a needleless access system as described herein.

Multi-use dispensing vessels in accordance with the present invention contain formulations suitable for parenteral use, e.g., intravenous administration. Such vessels are commercially available and include the needleless access systems such as the InterLink™ I.V. Access System by Becton Dickinson, and SmartSite System by Alaris Medical Systems (see, also various needless transfer system in U.S. Pat. No. 6,045,538; U.S. Pat. No. 5,716,346; U.S. Pat. No. 6,406,455; U.S. Pat. No. 5,360,413; U.S. Pat. No. 5,549,577; WO 01/68017; WO 02/34198). Thus, it is understood that the formulation of the present invention is formulated for multi-use vessels, including in particular, those with needless access systems. Further, the formulation is suitably packaged with an insert from a governmental regulatory agency approving the multi-use of the vessel and formulation. In one embodiment, the vessels in accordance with the present invention are typically larger than 2 mL, a size that is generally used for single dose formulations in single-use vials and ampoules. In another embodiment, the multi-vessel is a needless access vial.

Multi-use vessels containing formulations of the present invention can reduce the cost of providing such formulations. For example, a vitamin D formulation in a multi-use vessel in accordance with the present invention is suitable for use at a dialysis center, where, perhaps, 50 patients are dialyzed per day, can significantly reduce costs, i.e., one container versus 50 containers per day. Multi-use vessels containing a formulation in accordance with the present invention may be used in administering doses to a patient on a daily basis or an episodic basis, e.g., once to three times per week.

Formulations in accordance with the present invention also are suitable for use in plastic (blow-fill) containers. For use in such containers, the blow-fill container formulations contain a solubilizer (e.g., a polysubstrate) a lipophilic antioxidant (e.g., BHT) and a preservative, e.g., an anti-microbial or anti-bacterial agent. For example, benzyl alcohol is a suitable preservative at a concentration of about 0.5% to about 3% w/w. Blow-fill container formulations include about 0.05% to about 5% w/w non-ionic solubilizer, about 20 to about 2000 ppm lipophilic antioxidant and about 0.5% to about 20% preservative. A typical blow-fill container formulation includes 0.05% to 5% w/w TWEEN™-20, 20 to 2000 ppm BHT, and 0.5% to 3% benzyl alcohol. Blow-fill containers can be made with a luer lock entry, thereby enabling them to be connected directly to a syringe or an indwelling catheter of a patient. Blow-fill containers containing formulations in accordance with the present invention may be used to administer doses to patients on a daily bases or an episodic basis, e.g., once to three times per week.

Formulations may be readily prepared by using pharmacopeia grade reagents in which the reagents are made up in stock solutions from which the resulting solutions at the appropriate concentrations can be made. Once the appropriate amounts of stock solution are combined, it is often desirable to stir the reagents for several minutes under nitrogen gas gently blown over the top of the mixture, i.e., a nitrogen gas overlay. Degassed Water for Injection is then added to the desired final volume, and stirring under nitrogen gas continued for another several minutes.

Additionally, as described hereinabove, vitamin D compounds in accordance with the present invention include prodrugs, i.e., drugs that require further metabolic processing in vivo, e.g., additional hydroxlation. Such prodrugs of vitamin D compounds that have been found to be effective therapeutic agents are generally less reactive than, e.g., the dihydroxy natural hormone, 1α,25-dihydroxyvitamin D₃. These compounds offer further advantage for use in multi-use vessels.

In addition, formulations of the current invention may be terminally sterilized by means of, e.g., autoclaving.

A multi-use vessel in accordance with the present invention comprising a pharmaceutical formulation containing a vitamin D or a vitamin D analog like those substances described herein, is suitable for use in the treatment and/or prophylaxis of i) diseases or conditions characterized by abnormal cell differentiation and/or cell proliferation such as, e.g., psoriasis and other disturbances of keratinisation, neoplastic diseases and cancers, such as pancreas, breast, colon and prostate cancers as well as skin cancer; ii) diseases of, or imbalance in, the immune system, such as host-versus-graft and graft-versus-host reaction and transplant rejection, and auto-immune diseases such as discoid and systemic lupus erythematosus, diabetes mellitus and chronic dermatoses of auto-immune type, e.g., scleroderma and pemphigus vulgaris; iii) inflammatory diseases such as rheumatoid arthritis, as well as in the treatment and/or prophylaxis of a number of (iv) other diseases or disease states, including hyperparathyroidism, particularly secondary hyperparathyroidism associated with renal failure, and in (v) promoting osteogenesis and treating/preventing bone loss as in osteoporosis and osteosmalacia. (For use of vitamin D compounds for treatment and prophylaxis of various diseases/disorders, see, e.g., U.S. Pat. Nos. 5,9722,917; 5,798,345; 5,763,428; 5,602,116; 5,869,386; 5,104,864; 5,403,831; 5,880,114; 5,561,123.) The vitamin D formulations in accordance with the present invention are especially suited for treatment of cell proliferative disorders such as psoriasis; disorders of the calcium metabolism, such as osteomalacia; or neoplastic diseases, such as cancers of the pancreas, breast, colon or prostate, where the method of treatment comprises administering to a patient in need thereof in a dosage form of a multi-use vessel containing a formulation in accordance with the present invention. Daily dosages as well as episodic doses, e.g., once to three times per week, are contemplated.

The present invention is further explained by the following examples which should not be construed by way of limiting the scope of the present invention.

### Preparation of Stock Solutions

### EXAMPLE 1

### Doxercalciferol (1α-hydroxyvitamin D₂) Stock Solution

12.558 mg of doxercalciferol was weighed and transferred to a 10-mL volumetric flask. The solid was diluted to volume with ethanol and the flask was vigorously shaken to dissolve the solid.

### EXAMPLE 2

### Butylated Hydroxytoluene (BHT) Stock Solution

2.22 g BHT was transferred to a 100-mL volumetric flask. The solid was diluted to volume with ethanol and the flask was vigorously shaken to dissolve the solid.

### EXAMPLE 3

### 10% Tween™-20

100 g Tween™-20KR was transferred to a 1-L volumetric flask and diluted to volume with degassed Water for Injection. A magnetic stir bar was added and the mixture stirred to mix.

### Formulations

### EXAMPLE 4

### Doxercalciferol Formulations

The general procedure for preparing doxercalciferol formulations was as follows. To a glass formulation vessel was added Doxercalciferol Stock Solution, 10% Tween™-20, BHT Stock Solution, and ethanol, in the order listed. Nitrogen gas was gently blown over the top of the mixture. A stir bar was added to the mixture and stirred for not less than 20 minutes while continuing the nitrogen gas overlay. Degassed Water for Injection was added to bring the final volume to one liter. The mixture was stirred for not less than 20 minutes while continuing the nitrogen gas overlay. The volumes of each component used in preparing the formulations are listed in the Table 1 below.

TABLE 1
Preparation of Doxercalciferol Formulations

| Doxercalciferol | Tween ™ −20 | BHT Stock | Ethanol | Water for |
|---|---|---|---|---|
| Stock (mL) | Stock (mL) | (mL) | (mL) | Injection (mL) |
| 2.0 | 50 | 1.0 | 27 | 920 |
| 6.0 | 250 | 1.0 | 23 | 720 |

### EXAMPLE 5

### Multi-Use Multidose Vessel Formulation

A multi-dose formulation of calcitriol is prepared similarly to the methods described in Examples 1-4. Calcitriol is present at a concentration of greater than 2 μg in greater than 2 mL

A multidose formulation of doxercalciferol is prepared in a manner as described above in which doxercalciferol is present at a concentration of greater than 4 μg in greater than 2 mL

A multidose formulation of paracalcitriol is prepared in a manner similar to the methods described above in Examples 1-4. Paracalcitol is present at a concentration of greater than 10 μg in greater than 2 mL

### EXAMPLE 6

### Blow-Fill Container Formulation

Doxercalciferol, BHT and TWEEN™-20 stock solutions are made similar to Examples 1-3. These stock solutions are used to prepare specific solutions suitable for blow-fill containers similar to Example 4, except benzyl alcohol is used to prepare the formulation and has final concentrations of 0.5% to 3% w/w.

In summary, the present invention provides an improved formulation for vitamin D compounds that are only slightly soluble in an aqueous vehicle. The formulation in addition to the vitamin D includes a lipophilic antioxidant, a non-ionic solubilizer or surfactant, and optionally, an agent that is an organic solvent, a preservative or both. The formulation in accordance with the present invention is suitable for use in multi-use dispensing vessels and in plastic (blow-fill) containers.

All patents, publications and references cited herein are hereby fully incorporated by reference. In case of conflict between the present disclosure and incorporated patents, publications and references, the present disclosure should control.

While the present invention has now been described and exemplified with some specificity, those skilled in the art will appreciate the various modifications, including variations, additions, and omissions, that may be made in what has been described. Accordingly, it is intended that these modifications also be encompassed by the present invention and that the scope of the present invention be limited solely by the broadest interpretation that lawfully can be accorded the appended claims.

What is claimed is:

1. A multi-use vessel comprising a pharmaceutical formulation and a package insert including instructions for use and governmental regulatory agency approval for multi-use, the formulation comprising an active vitamin D compound, a non-ionic solubilizer, a lipophilic antioxidant and an aqueous vehicle.

2. A vessel as set forth in claim 1 further comprising a needleless access vial.

3. A vessel as set forth in claim 2, wherein the vial has a volume greater than 2 mL and the formulation has a volume of greater than 2 mL.

4. A vessel as set forth in claim 3, wherein the vitamin D includes an active vitamin D which includes a vitamin D precursor or a vitamin D metabolite.

5. A vessel as set forth in claim 4, wherein the vitamin D includes doxercalciferol, seocalcitol, maxacalcitol, falecalcitriol, calcitriol, calcipotriol and paricalcitol.

6. A vessel as set forth in claim 5, wherein the vitamin D compound includes doxercalciferol.

7. A vessel as set forth in claim 5, wherein the vitamin D compound includes seocalcitol.

8. A vessel as set forth in claim 5, wherein the vitamin D compound includes calcitriol.

9. A vessel as set forth in claim 5, wherein the vitamin D compound includes paricalcitol.

10. A vessel as set forth in claim 5, wherein the vitamin D compound includes calcipotriol.

11. A vessel as set forth in claim 5, wherein the vitamin D compound includes maxacalcitol.

12. A vessel as set forth in claim 5, wherein the vitamin D compound includes falecalcitriol.

13. A vessel as set forth in claim 1, wherein the non-ionic solubilizer includes polyoxyalkylenes, polysorbates, fatty acid esters of glycerol, cyclodextrins, dextrans, fatty alcohol ethers of oligoglucosides and polyoxyethylene type compounds.

14. A vessel as set forth in claim 13, wherein the non-ionic solubilizer is a polysorbate.

15. A vessel as set forth in claim 1, wherein the lipophilic antioxidant includes butylated hydroxytoluene (BHT), lipoic acid, lycopene, lutein, lycophyll, xanthophyll, carotene, zeaxanthin or vitamin E and esters thereof.

16. A vessel as set forth in claim 15, wherein the lipophilic antioxidant is BHT.

17. A vessel as set forth in claim 13, wherein the non-ionic solubilizer is present at a concentration of about 0.05% to about 5% w/w.

18. A vessel as set forth in claim 15, wherein the lipophilic antioxidant is present at a concentration of about 20 to about 2000 ppm.

19. A vessel as set forth in claim 3, wherein the vitamin D is administered in a dose of about 2 μg to about 100μ/dose.

20. A vessel as set forth in claim 3, wherein the formulation is suitable for patenteral administration.

21. A vessel as set forth in claim 3, wherein the formulation is administered on an episodically.

22. A vessel as set forth in claim 21, wherein the formulation is administered one to three times per week.

23. A vessel as set forth in claim 3, wherein the formulation is administered on a daily basis.

24. A vessel as set forth in claim 3, wherein the formulation further comprises an optional agent.

25. A vessel as set forth in claim 24, wherein the optional agent includes an alcohol.

26. A vessel as set forth in claim 25, wherein the alcohol is present at a concentration of 0% to about 30% w/w.

27. A vessel as set forth in claim 25, wherein the alcohol includes ethanol.

28. A vessel as set forth in claim 25, wherein the alcohol includes benzyl alcohol.

29. A multi-use vessel comprising a needleless access vial and a vitamin D formulation, the formulation comprising an active vitamin D compound, a non-ionic solubilizer, a lipophilic agent, an organic solvent and an aqueous vehicle, the vial and formulation having volumes greater than 2 mL.

30. A vessel comprising a greater than 2 mL, multi-use, needleless access vial containing a vitamin D formulation, and a package insert including instructions for use and approval from a regulatory agency for multi-use.

31. A vessel as set forth in claim 30, wherein the formulation includes doxercalferol.

32. A vessel as set forth in claim 30, wherein the formulation includes paricalcitol.

33. A vessel as set forth in claim 30, wherein the formulation includes calcitriol.

34. A vessel as set forth in claim 30, wherein the formulation includes seocalcitol.

35. A vessel as set forth in claim 30, wherein the formulation includes calcipotriol.

36. A vessel as set forth in claim 30, wherein the formulation includes maxacalcitol.

37. A vessel as set forth in claim 30, wherein the formulation includes falecalcitriol.

38. A vessel as set forth in claim 30, wherein the formulation includes about 0.05% to about 5% w/w non-ionic solubilizer, about 20 to about 2000 ppm lipophilic antioxidant, and 0% to about 30% organic solvent.

39. A vessel as set forth in claim 38, wherein the formulation includes about 0.5% to about 5% w/w/TWEEN™-20, 20 ppm BHT, and 2.5% w/w benzyl alcohol.

40. A method of treating diseases or disorders which include hyperproliferative diseases, disorders of calcium metabolism, and hyperparathyroidism, comprising parenterally administering to a mammal in need thereof a dosage form comprising the multi-use vessel of claim 1.

41. A method as set forth in claim 40, wherein the hyperproliferative diseases are cancers of the breast, colon, prostate and pancreas.

42. A method as set forth in claim 40, wherein the hyperproliferative disease is psoriasis.

43. A method as set forth in claim 40, wherein the hyperparathyroidism is secondary hyperparathyroidism.

44. A method as set forth in claim 40, wherein the disease or disorder is disorders of calcium metabolism.

45. A vessel comprising a plastic, blow-fill vial containing a vitamin D formulation, and a package insert including instructions for use and marketing approval from a governmental regulatory agency.

46. A vessel as set forth in claim 45 further comprising a luer lock.

47. A vessel as set forth in claim 45, wherein doses of the formulation are administered episodically.

48. A vessel as set forth in claim 47, wherein the formulation is administered once per week to three times per week.

49. A vessel as set forth in 45, wherein doses of the formulation are administered on a daily basis.

50. A method of treating a subject with an active vitamin D compound, comprising administering parenterally to the subject a dosage form comprising the vessel of claim 45.

51. A multi-use vessel comprising a pharmaceutical formulation and a package insert including instructions for use and governmental regulatory agency approval for multi-use, the formulation comprising an active vitamin D compound.
